SEQUENCE LISTING (2 sequences)

SEQ ID NO: 1 - protein, 11 aa, Homo sapiens
RPKPQQFFGLM

SEQ ID NO: 2 - protein, 36 aa, Homo sapiens
YPSKPDNPGEDAPAEDMARYYSALRHYINLITRQRY

(19) United States
(12) Patent Application Publication
(10) Pub. No.: US 2014/0234878 A1
(43) Pub. Date: Aug. 21, 2014

(54) COMPOSITION FOR DIAGNOSING ACUTE MYOCARDIAL INFARCTION, KIT FOR DIAGNOSING ACUTE MYOCARDIAL INFARCTION COMPRISING SAME, AND METHOD OF DIAGNOSING ACUTE MYOCARDIAL INFARCTION

(71) Applicant: KOREA INSTITUTE OF SCIENCE AND TECHNOLOGY, Seoul (KR)

(72) Inventors: Min-Jung KANG, Seoul (KR); Hyojeong HAN, Seoul (KR); Byung Hwa JUNG, Seoul (KR)

(73) Assignee: KOREA INSTITUTE OF SCIENCE AND TECHNOLOGY, Seoul (KR)

(21) Appl. No.: 13/947,253

(22) Filed: Jul. 22, 2013

(30) Foreign Application Priority Data
Feb. 21, 2013 (KR) 10-2013-0018839

(51) Int. Cl. G01N 33/68; C07K 16/18

(52) U.S. Cl. CPC G01N 33/6893; C07K 16/18
USPC 435/7.94; 530/387.9; 436/501; 435/7.92; 435/7.95

(57) ABSTRACT

Provided are compositions for diagnosing acute myocardial infarction including a preparation for measuring an amount of a substance P (SubP) or neuropeptide Y (NpY), kits for diagnosing acute myocardial infarction comprising the composition, and methods of diagnosing acute myocardial infarction based on the amount of the substance P or the neuropeptide Y.

### CROSS-REFERENCE TO RELATED PATENT APPLICATION

This application claims the benefit of Korean Patent Application No. 10-2013-0018839, filed on Feb. 21, 2013, in the Korean Intellectual Property Office, the entire disclosure of which is hereby incorporated by reference.

### BACKGROUND OF THE INVENTION

1. Field

The present disclosure relates to a composition for diagnosing acute myocardial infarction, a kit for diagnosing acute myocardial infarction including the composition, and a method of diagnosing acute myocardial infarction by using the same.

2. Description of the Related Art

Drastic changes in dietary habits along with rapid industrialization in Korea has resulted in an increase in chronic degenerative diseases in Korea, in particular, the rate of ischemic coronary disease among circulatory diseases. Over the last decade, the death rate due to circulatory diseases such as cerebrovascular diseases and cardiovascular diseases has been decreasing. However, the death rate due to ischemic heart disease such as acute myocardial infarction increased from 13.3 per 100,000 people in 1993 to 24.6 per 100,000 people in 2003, i.e., 11.3 per 100,000 for ten years (the Statistics Korea, 2004).

Acute myocardial infarction is a disease caused by rapid progress of myocardial ischemia and irreversible necrosis due to ruptures in atheromatous plaque in the coronary artery which blocks the coronary artery due to an acute occlusive thrombus. Acute myocardial infarction, once occurred, can lead to death in up to 30% of afflicted people within 30 days, and more than half of them may die before they arrive at a hospital. Over the last two decades, the death rate due to acute myocardial infarction has decreased by about 30%. However, one of 25 patients with acute myocardial infarction, who had been hospitalized due to its symptoms, still dies within one year after they are discharged from the hospital. Major factors associated with acute myocardial infarction include age, male, hypertension, diabetes, hyperlipidemia, arterial diseases in the family history and smoking, etc. In subjects with myocardial infarction, rapid diagnosis and accurate prediction are essential.

Conventionally, myocardial infarction has been diagnosed by electrocardiography, and observation of difference in hormonal concentrations (CK-MB, Myoglobin, Troponin I) via blood tests. Since CK-MB, Myoglobin, and Troponin I proteins, at the time of acute myocardial infarction, flow into blood thereby increasing their concentration rapidly, acute myocardial infarction is diagnosed by their blood concentraion. However, their specificity is about 60% to 70% and thus it is also probable that the blood tests may come out negative even for people with acute myocardial infarction. One well-known marker associated with cardiovascular diseases is N-terminal proBNP (NT-proBNP). The concentration of NT-proBNP in plasma is known to increase over time in subjects with acute myocardial infarction, but has also been studied as a marker for the diagnosis of congestive heart failure, CHF. Accordingly, there is a need for the development of a marker with increased specificity and sensitivity for diagnosing acute myocardial infarction.

### SUMMARY

The disclosure provides a composition for diagnosing acute myocardial infarction, the composition including a preparation for measuring an amount of substance P (SubP) or neuropeptide Y (NpY).

Also, the disclosure provides a kit for diagnosing acute myocardial infarction including the composition.

Additionally the disclosure also provides a method of diagnosing acute myocardial infarction based on the amount of SubP or NpY. According to an aspect of the present invention, there is provided a composition including a preparation for measuring the amount of a SubP or NpY protein for the diagnosis of acute myocardial infarction.

### BRIEF DESCRIPTION OF THE DRAWINGS

These and/or other aspects will become apparent and more readily appreciated from the following description of the embodiments, taken in conjunction with the accompanying drawings in which:

FIG. 1 includes two panels (FIGS. 1A and 1B). FIGS. 1A and 1B are a schematic diagram illustrating the method of enzyme-linked immunosorbent assay (ELISA) assay using monoclonal antibodies specific to SubP and NpY peptides, respectively;

FIG. 2 includes three panels (FIGS. 2A-2C). FIGS. 2A-2C are graphs of calibration curves in which standard materials of NT-proBNP, SubP, and NpY were analyzed using an ELISA kit, wherein FIG. 2a is a calibration curve for SubP, FIG. 2b is a calibration curve for NT-proBNP, and FIG. 2c is a calibration curve for NpY, respectively;

FIG. 3 includes nine panels (FIGS. 3A-3I). FIGS. 3A-3C, 3D-3F, and 3G-3I are respectively graphs of concentrations of NT-proBNP, SubP and NpY analyzed using blood samples of normal people and those of patients with a cardiovascular disease. FIGS. 3A, 3D, and 3G are graphs of a case for acute myocardial infarction (AMI); FIGS. 3B, 3E, and 3H are graphs of a case for unstable angina (UA); and FIGS. 3C, 3F, and 3I are graphs of a case for angina pectoris (AP).

FIG. 4 includes nine panels (FIGS. 4A-4I). FIGS. 4A-4C, 4D-4F, and 4G-4I are respectively graphs of a Receiver Operating Characteristic (ROC) of NT-proBNP, SubP and NpY obtained using blood samples of normal people and those of subjects with a cardiovascular disease. FIGS. 4A, 4D, and 4G are graphs of a case for acute myocardial infarction (AMI); FIGS. 4B, 4E, and 4H are graphs of a case for unstable angina (UA); and FIGS. 4C, 4F, and 4I are graphs of a case for angina pectoris (AP).

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

According to an embodiment of the present invention, there is provided composition for diagnosing acute myocardial infarction including a preparation for measuring an amount of substance P (SubP) or neuropeptide Y (NpY).

The term, “substance P (SubP)” used herein, refers to “tackykinin”, which is a mammalian neuropeptide. The protein sequence of the SubP is represented as SEQ ID NO: 1.

The term, “neuropeptide Y (NpY)” used herein, refers to a peptide, which is widely distributed in the central nervous system in mammals. The protein sequence of NpY is represented as SEQ ID NO: 2.

TABLE 1

| Peptide | Amino acid sequence | SEQ ID NO |
|---|---|---|
| SubP | RPKPQQFFGLM | SEQ ID NO: 1 |
| NpY | YPSKPDNPGEDAPAEDMARYYSALRHY | SEQ ID NO: 2 |
| | INLITRQRY | |

The term, “diagnosis” used herein, refers to the identification of the presence or characteristics of pathophysiology. In the present disclosure, the diagnosis refers to identification of the presence of acute myocardial infarction.

The term, “measuring the amount of” a protein used herein, refers to the identification of the presence of the protein and their amounts using a sample obtained from a subject for the diagnosis of acute myocardial infarction. The term, “the preparation for measuring the amount of” a protein used herein, refers to a protein which may be used for the detection and/or quantification of proteins by identifying the amount of the protein depending on acute myocardial infarction.

The preparation for measuring the amount of proteins may be antibodies which specifically bind to SubP or NpY or a fragment thereof. The term, “antibodies” used herein, is well known in the art, and refers to a immunoglobulin specific to an antigenic domain. The antibodies in the present disclosure refer to the antibodies which bind specifically to SubP or NpY, or a fragment thereof, and they can be prepared from SubP or NpY, or a fragment thereof according to a conventional method in the art. Types of the antibodies include polyclonal antibodies, monoclonal antibodies, and recombinant antibodies, and all the immunoglobulin antibodies. The antibodies may be in a complete form of having two full-length light chains and two full-length heavy chains. Additionally, the antibodies may include specific antibodies including humanized antibodies, etc.

The preparation for measuring the amount of proteins may be partial peptides having a domain which specifically bind to SubP or NpY or a fragment thereof. The term, “peptides having a specific binding domain” used herein, refers to a specific antigen binding domain instructed by an antigenic domain although not with a complete structure of an antibody, that is, a polypeptide with a binding domain. The partial peptide includes a functional fragment of an antibody molecule instead of a complete form of an antibody having two light chains and two heavy chains. The functional fragment of an antibody molecule refers to a fragment having at least an antigen binding function, and it includes Fab, F(ab′), F(ab′)2 and Fv, etc. The partial peptide may have at least 7 amino acids, for example, at least 9 amino acids, or for example, at least 12 amino acids.

The method of measuring the amount of protein expression may include, for example, western blot, enzyme linked immunosorbent assay (ELISA), radioimmunoassay (RIA), radioimmunodiffusion, Ouchterlony immunodiffusion, rocket immunoelectrophoresis, histoimmunostaining, immunoprecipitation assay, complement fixation test, fluorescence-activated cell sorting (FACS), protein chips or a combination thereof.

The measurement of the amount of the proteins may be performed, for example, via enzyme-linked immunosorbent assay (ELISA). There are various types of ELISA available for the measurement; e.g., a direct ELISA which uses antigens adhered to a solid support and labeled antibodies that can specifically bind to the antigens; an indirect ELISA, which uses antigens adhered to a solid support, primary antibodies that can specifically bind to the antigens, and labeled secondary antibodies that can specifically bind to the primary antibodies; a direct sandwich ELISA, which uses antigens adhered to a solid support, primary antibodies that can specifically bind to the antigens, and other labeled antibodies which recognize antigens in the complex of the primary antibodies and the antigens; an indirect sandwich ELISA, which uses capture antibodies adhered to a solid support and binding to antigens, primary antibodies that can specifically bind to the antigens, labeled secondary antibodies that can specifically bind to the primary antibodies, etc.

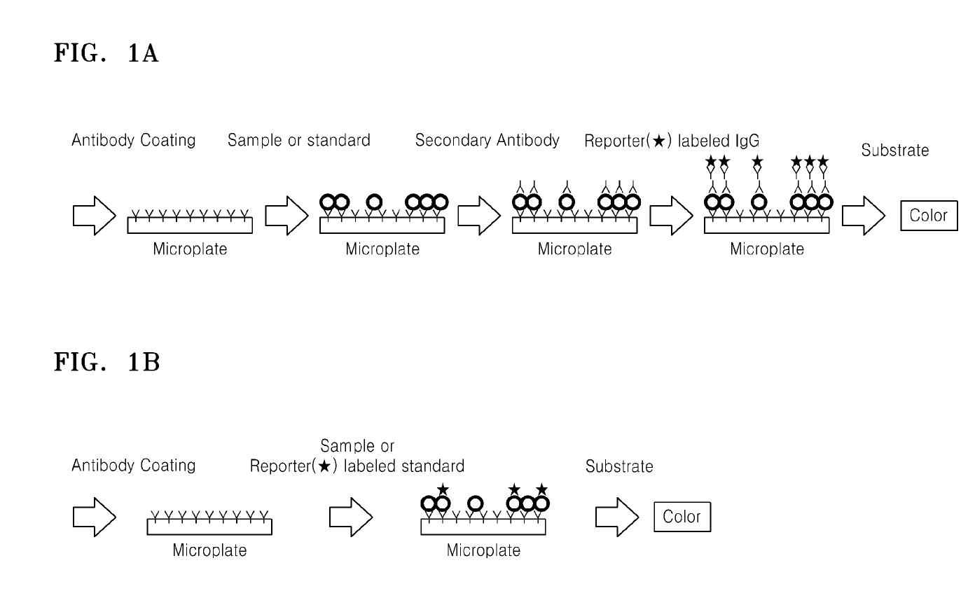

For example, when the sandwich ELISA is used, the method may include; coating antibodies, which specifically bind to SubP or NpY protein or a fragment thereof as primary antibodies, to the surface of a solid substrate; inducing an antigen-antibody reaction by allowing a respective blood sample obtained from a normal subject and a sample obtained from a subject suspected of having an acute myocardial infarction to contact with the antibodies; reacting the resultant of the induced antibody-antigen reaction with secondary antibodies linked with an enzyme; and detecting the activity of the enzyme (FIG. 1A).

The solid substrate may include hydrocarbon polymer (e.g., polystyrene and polypropylene), glass, metal or gel, and for example, may include, for example, a microtiter plate. The enzyme to be linked to the secondary antibodies may include those enzymes that can catalyze a color reaction, a fluorescent reaction, a luminescent reaction, or an infrared reaction, for example, alkaline phosphatase, β-galactosidase, horseradish peroxidase, luciferase, or cytochrome P450. When alkaline phosphatase is used as an enzyme to be linked to the secondary antibodies, brown reaction substrates such as bromochloroindolyl phosphate (BCIP), nitroblue tetrazolium (NBT), naphthol-AS-B1-phosphate and enhanced chemifluorescence (ECF) may be used. When horseradish peroxidase is used, substrates such as chloronaphthol, aminoethylcarbazol, diaminobenzidine, D-luciferin, lucigenin (bis-N-methylacridiniumnitrate), resorufin benzyl ether, luminol, Amplex® red reagent (10-acetyl-3,7-dihydroxyphenoxazine), p-phenylenediamine-HCl and pyrocatechol (HYR), tetramethylbenzidine (TMB), 2,2′-azino-bis[3-ethylbenzothiazoline sulfonate] (ABTS), o-phenylenediamine (OPD) and naphthol/pyronine, glucose oxidase and nitroblue tetrazolium (t-NBT) and phenzaine methosulfate (m-PMS), etc.

When the measurement of the amount of a protein depends on the direct ELISA, the method may include: coating antibodies, which specifically bind to SubP or NpY protein or a fragment thereof as primary antibodies, to the surface of a solid substrate; inducing an antigen-antibody reaction by allowing the respective blood sample obtained from a normal subject and a sample obtained from a subject suspected of having an acute myocardial infarction and a certain amount of a labeled standard substance (a peptide standard to which an enzyme, an organic fluorescent material, or a nanofluorescent material is linked) to competitively contact with the antibodies; measuring the resultant of the induced antibody-antigen reaction via a microplate reader or a fluorescence reader; and directly detecting the concentration of the protein (FIG. 1B).

The measurement of the amount of protein expression may be performed, for example, by western blot analysis using at least one antibodies. The total proteins are separated from the sample, and electrophoresed to separate them according to their size, transferred to a nitrocellulose membrane and reacted with antibodies. The amount of the protein is measured by the amount of the generated antigen-antibody complex using labeled antibodies and thus, the presence of acute myocardial infarction may be detected.

The detection method may be performed by examining the amount of the protein in the normal control group and the amount of the protein in the biological sample to be tested. The amount of the protein may be represented by an absolute (e.g., μg/Ml) or relative (e.g., relative intensity of a signal) difference in the protein between the normal control group and the biological sample to be tested.

In another aspect of the present invention, there is provided a kit for the diagnosis of acute myocardial infarction including the above-described composition.

The diagnostic kit for acute myocardial infarction may diagnose acute myocardial infarction by measuring the amount of SubP or NpY protein. The diagnostic kit for acute myocardial infarction may include a preparation for measuring the amount of SubP or NpY protein, for example, antibodies which specifically bind to SubP or NpY, or a fragment thereof; partial peptides having a domain which specifically bind to SubP or NpY, or a fragment thereof; or a combination thereof. In addition, the kit may include at least one component of the composition suitable for the analysis method of measuring the amount of protein expression used by the kit, a solution or a device.

In an embodiment of the present invention, the kit may include antibodies which specifically bind to SubP or NpY or a fragment thereof, a substrate for the immunological detection of antibodies, a suitable buffer solution, secondary antibodies labeled with a chromogenic enzyme or fluorescent material, etc. The substrate to be used may include a nitrocellulose membrane, a 96-well plate synthesized by polyvinyl resin, a 96-well plate synthesized by polystyrene resin, slide glass, etc. The chromogenic enzyme to be used may include alkaline phosphatase, and the fluorescent material may include FITC, RITC, etc. The chromogenic substrate to be used may include 2,2′-azino-bis[3-ethylbenzothiazoline sulfonate] (ABTS), o-phenylenediamine (OPD) and tetramethyl benzidine (TMB) or a combination thereof.

In another aspect of the present invention, there is provided a method of diagnosing acute myocardial infarction including:

measuring an amount of substance P or neuropeptide Y in a sample obtained from a subject;

comparing the measured amount of the substance P or the neuropeptide Y with that of a normal control group; and

determining the subject as having acute myocardial infarction if the amount of the protein in the sample obtained from the subject is higher than that of the normal control group.

The method may include measuring the amount of protein expression of SubP or NpY protein in the sample obtained from the subject.

The term, “subject” used herein, refers to a subject suspected of having an acute myocardial infarction, and includes any subject in whom the amount of SubP or NpY protein may vary due to the acute myocardial infarction.

The term, “sample” used herein, includes blood, liquid bone marrow, lymph, saliva, lachrymal fluid, mucus, amniotic fluid, or a combination thereof.

The term, measuring the amount of” a protein used herein, refers to a process of identifying the presence of a protein, and the amount of its expression for the diagnosis of acute myocardial infarction. The method includes, for example, western blot, ELISA, radioimmunoassay (RIA), radioimmunodiffusion, Ouchterlony immunodiffusion, rocket immunoelectrophoresis, histoimmunostaining, complement fixation test, FACS, protein chips or a combination thereof.

The method may include comparing the measured amount of protein expression with that of a normal control group. The comparison may be performed by examining the amount of the protein expression in the normal control group and the amount of the protein expression in the sample obtained from a subject. The amount of the protein may be represented by an absolute (e.g., μg/Ml) or relative (e.g., relative intensity of a signal) difference in the protein between the normal control group and the biological sample to be tested.

The method may include diagnosing the subject as having acute myocardial infarction if the amount of the protein in the sample obtained from the subject is higher than that of the normal control group.

The method diagnoses the subject as having acute myocardial infarction when the amount of SubP is 53.58 pg/ml blood or more, and the amount of NpY protein is 525 pg/ml blood or more; diagnoses the subject as having unstable angina when the amount of SubP is less than 53.58 pg/ml blood, and the amount of NpY protein is 407 pg/ml blood or more but less than 525 pg/ml blood; and diagnosed as not having acute myocardial infarction or unstable angina when the amount of SubP is less than 53.58 pg/ml blood, and the amount of the neuropeptide Y is less than 407 pg/ml blood.

In an aspect of the present invention, there are provided a composition for the diagnosis of acute myocardial infarction, a kit, and a method of the diagnosis which can be useful for the diagnosis of acute myocardial infarction of subjects suspected of having a cardiovascular disease.

### EXAMPLES

### Example 1

### Preparation of Blood Samples from Patients with a Cardiovascular Disease

500 μl each of blood samples of normal people and patients with a cardiovascular disease obtained in Korea University College of Medicine were respectively diluted twofold with 10% (v/v) formic acid buffer, and then peptides were extracted using an Oasis® HLB 1 cc (30 mg) Extraction Cartridges Solid phase Extraction Kit (Waters, Ireland). Meanwhile, intrinsic peptides in blood serum and blood plasma were separated from the proteins and extracted using a 30 kDa Molecular cut-off filter (Millipore, USA). A solution containing the extracted peptide was dried using a N2 Evaporator and Freeze-Dryer, and dissolved in 50 μl of assay buffer (provided by each enzyme-linked immunosorbent assay (ELISA) manufacturer) for immunological analysis, or 25 μl of blood serum and blood plasma were diluted two fold in PBS buffer and directly analyzed. For LC-MS/MS analysis, the solution was dissolved in 100 μl of 0.2% (v/v) formic acid in 50% (v/v) methanol and analyzed.

### Example 2

### ELISA Assay in Blood Sample

### 2.1 Detection of Acute Myocardial Infarction Using NT-proBNP

NT-proBNP, which is known to a biomarker for heart diseases, was used to a control group to compare experimental results. Experiments were performed using a total of four types of blood sera of subjects, i.e., from 29 normal people, and 30, 28 and 30 patients with acute myocardial infarction, unstable angina, and angina pectoris, respectively. A kit purchased from BIOMEDICA was used for an ELISA experiment, and ELISA assay was performed based on a sandwich binding principle. The concentration of a standard solution for each calibration curve was 0, 10, 40, 160, 640 fmol/ml, respectively. Human NT-proBNP was used as a comparison group. Then, each well of a microplate coated with polyclonal sheep anti NT-proBNP antibodies was added in duplicate with 50 μl each of standard solution and the control group, respectively, and added in triplicate with 50 μl of four types of blood sera of subjects. Then, 200 μl of secondary antibodies (sheep anti-human NT-proBNP-HRP) conjugated with horseradish peroxidase (HRP) was added into each well and incubated at room temperature for 3 hours. Each well was then washed 5 times with washing buffer, and 200 μl of a substrate (TMB solution) was added into each well, and incubated at room temperature in darkness for 8 minutes. Then, 50 μl of a stop solution (IM sulfuric acid) to each well was added, and an absorbance of the resultant in each well was measured at 450 nm by using a microplate reader.

### 2.2 Detection of Acute Myocardial Infarction Using SubP

Experiments were performed using a total of four types of blood sera of subjects, i.e., from 29 normal people, and 30, 29 and 30 patients with acute myocardial infarction, unstable angina, and angina pectoris, respectively. An R&D system kit was used for an ELISA experiment, and an ELISA assay was performed based on a competitive binding principle. The concentration of a standard solution for each calibration curve was prepared by diluting 50 μl of SubP standard solution (50 ng/ml) into 1 Ml Calibrator Diluent RD5-45 (R&D systems®), and then continuously diluting 300 μl of SubP standard solution (50 ng/ml) into 1 Ml Calibrator Diluent RD5-45 (R&D systems®) thereby obtaining the standard solution at concentrations of 2500, 1250, 625, 312, 156, 78, 39, and 0 pg/Ml, respectively. As a comparison group, 213-540, 517-982, and 1224-2217 pg/Ml of control group and zero standard wells were used. Standard solution, control group and zero standard well preparations were all performed in duplicate. Blood sera samples were diluted twofold with 80 μl of sample and 80 μl of Calibrator Diluent RD5-45 and experimented in triplicate. Wells of an ELISA microplate (provided with the kit) coated with anti-mouse polyclonal antibodies (R&D systems®) obtained by introducing SubP, an artificial peptide, to a goat were added in duplicate with 50 μl of standard solution and control group solution, respectively, and added in triplicate with 50 μl of four types of blood sera of subjects. Then, each well was added with 50 μl of primary antibody solution (murine monoclonal antibodies of SubP) and 50 μl of HRP conjugated with SubP in sequence, covered with an adhesive strip, and then incubated at room temperature in the stirrer of the microplate at 500±50 rpm for 3 hours. Each well was then washed 4 times with washing buffer, and 200 μl of a substrate (TMB solution) was added into each well, and incubated at room temperature for 30 minutes. Then, 50 μl of a stop solution (2N sulfuric acid) to each well was added, and upon observing the change of the solution in each well from blue to yellow, absorbance of the resultant in each well was measured at 450 nm by using a microplate reader.

### 2.3 Detection of Acute Myocardial Infarction Using NpY

Experiments were performed using a total of four types of blood sera of subjects, i.e., from 29 normal people, and 30, 28 and 29 patients with acute myocardial infarction, unstable angina, and angina pectoris, respectively. A kit purchased from PHOENIX PHARMACEUTICALS, INC. was used for an ELISA experiment, and an ELISA assay was performed based on a competitive binding principle. The concentration of a standard solution for each calibration curve was prepared by serial dilution of 1000 ng/Ml of a stock solution with 1× assay buffer solution at a concentration of 100, 10, 1, 0.1, and 0.01 ng/Ml, respectively. As a comparison group, 50 μl of a positive control group was added into each well, a control group and standard solution were added in duplicate, and a sample diluted threefold was added in triplicate into each well of a microplate. Then, 25 μl of primary antibodies (rabbit anti-peptide IgG) obtained by introducing artificial NpY into a rabbit (PHOENIX PHARMACEUTICALS, INC.) and 25 μl of biotinized peptide were added into each well, and incubated at room temperature in the stirrer of the microplate at 300-400 rpm for 2 hours. Each well was then washed 4 times with a washing buffer, and 100 μl of Streptavidin-horseradish peroxidase was added into each well, and stirred at 300-400 rpm at room temperature for 1 hour. Likewise, each well was washed 4 times with a washing buffer, and 100 μl of a substrate solution (TMB solution) was added into each well, and stirred at 300-400 rpm at room temperature for 1 hour. Then, 100 μl of a stop solution (2N HCl) to each well was added, and absorbance of the resultant in each well was measured at 450 nm by using a microplate reader.

### Example 3

### Detection of Acute Myocardial Infarction Using HPLC-MS/MS

500 μl each of blood samples of normal people or patients with a cardiovascular disease were diluted in 10% (v/v) formic acid buffer, and peptides were extracted therefrom using an Oasis® HLB 1 cc (30 mg) Extraction Cartridges Solid phase Extraction Kit (Waters, Ireland). Meanwhile, intrinsic peptides in blood serum and blood plasma were separated from the proteins and extracted using a 30 kDa Molecular cut-off filter (Millipore, USA). A solution containing the extracted peptide was dried using a N2 Evaporator and Freeze-Dryer, and then dissolved in 100 μl of 0.2% (v/v) formic acid in 50% (v/v) methanol and analyzed.

As an alternative, the peptide extracted from the prepared blood samples and concentrated was analyzed along with a peptide standard with known concentrations (0.01, 0.1, 1, 10 and 100 ng/ml) via multi reaction monitoring (MRM) method of LC-MS/MS (LTQ-Orbitrap, Thermo Scientific) thereby obtaining calibration curves based on chromatogram areas calculated using a standard solution, and by using the same, BNP (1-46), SubP, and NpY concentrations in blood were calculated. The conditions used for LC separation and MRM analysis are shown below.
mobile phase:

A: 10% acetonitrile in dH₂O including 0.2% formic acid

B: 10% dH₂O in acetonitrile with 0.2% formic acid
flow rate: 0.2 ml/min
gradient: B 0% [symbol] 0% (2 min), 0% [symbol] 20% (23 min), 20% [symbol] 35% (5 min), 35% [symbol] 40% (8 min), 40% [symbol] 70% (10 min), 70% [symbol] 100% (5 min, hold 5 min), 100% [symbol] 100% (2 min)
Multiple reaction monitoring (MRM) conditions for detecting peptides are shown in Table 2 below.

TABLE 2

| Peptide | Parent ion | Monitoring fragment ion |
|---|---|---|
| BNP (1-46) | 836 | 1047 |
| SubP | 674 | 656.5 |
| NpY | 856 | 950 |

### Example 4

### Confirmation of NT-proBNP, SubP and NpY in Patients with Acute Myocardial Infarction Using ELISA Assay

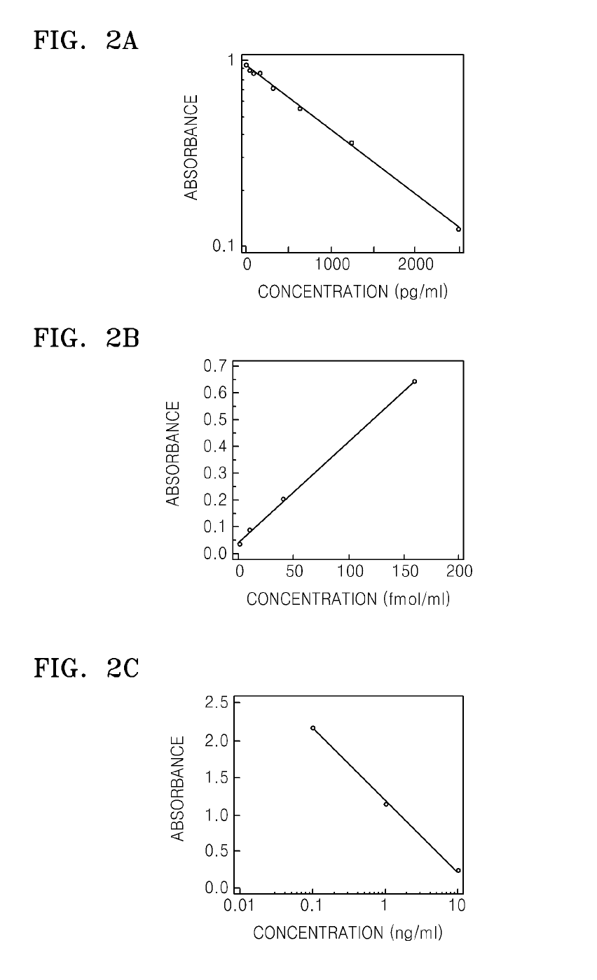

Calibration curves were plotted in order to analyze the concentrations of NT-proBNP, SubP and NpY in blood samples of normal people and patients with acute myocardial infarction using an ELISA assay. FIGS. 2A-2C are respectively graphs of calibration curves in which the standard materials of NT-proBNP, SubP, and NpY were analyzed using an ELISA kit. FIG. 2A is a calibration curve for SubP in which log(y)=−0.02897−0.0003464x and R²=0.99, FIG. 2B is a calibration curve for NT-proBNP in which, y=0.1732+0.002939x, and R²=0.99, and FIG. 2C is a calibration curve for NpY, in which y=1.1770−0.9695 log(x) and R²=0.99. As shown in FIGS. 2A-2C, all the substances had calibration curves with high linearity.

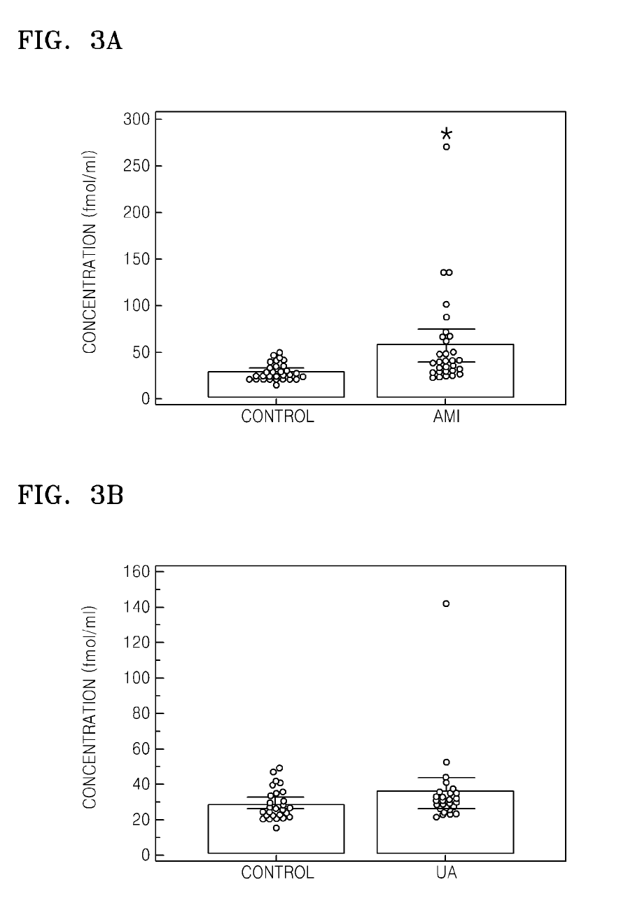

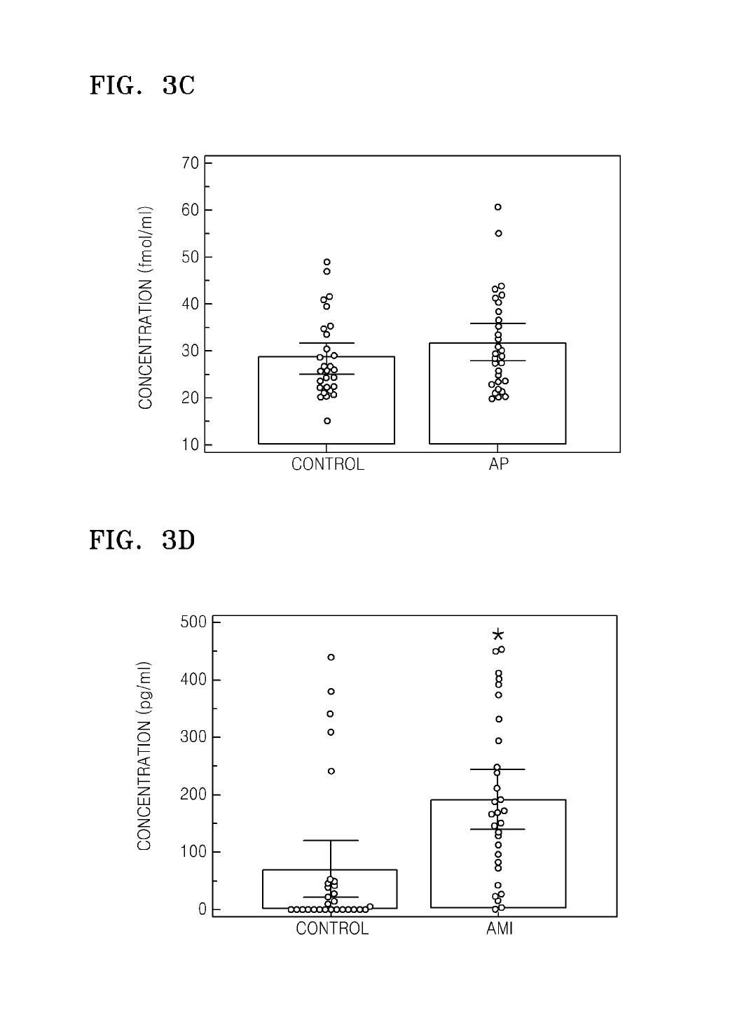

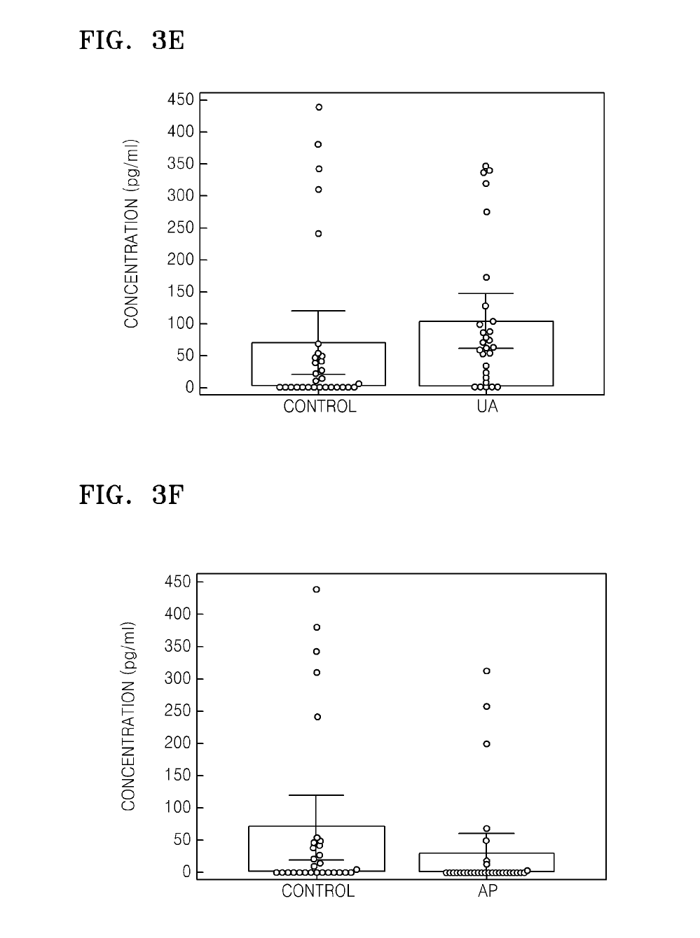

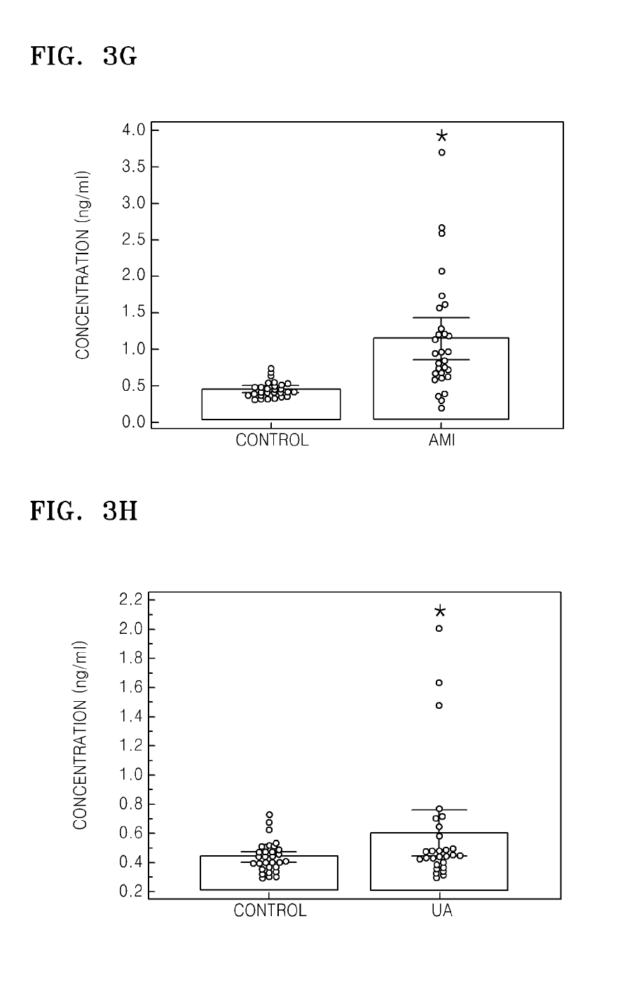

The blood samples of normal people and patients with acute myocardial infarction were analyzed using the calibration curves. FIGS. 3A-3C, 3D-3F, and 3G-3I are respectively graphs of analysis results of concentrations of NT-proBNP, SubP and NpY using blood samples of normal people and those of patients with a cardiovascular disease.

Referring to FIGS. 3A-3C, which relates to ‘NT-proBNP’, FIG. 3A represents a case for acute myocardial infarction (AMI), in which *P value=0.0036, and Cut-off value: >29.05 fmol/ml, FIG. 3B represents a case for unstable angina (UA), in which P value=0.1406, and Cut-off value: >26.62 fmol/ml, and FIG. 3C represents a case for angina pectoris (AP), in which P value=0.2029, and Cut-off value: >26.62 fmol/ml.

Referring to FIGS. 3D-3F, which relates to ‘SubP’, FIG. 3D represents a case for acute myocardial infarction (AMI), in which *P value=0.0011, and Cut-off value: >53.58 pg/ml, FIG. 3E represents a case for unstable angina (UA), in which P value=0.2993, and Cut-off value: >49.39 pg/ml, and FIG. 3F represents a case for angina pectoris (AP), in which P value=0.1730, and Cut-off value: <=3.32 pg/ml.

Referring to FIGS. 3G-3I, which relates to NpY, FIG. 3G represents a case for acute myocardial infarction (AMI), in which *P value=0.0002, and Cut-off value: >0.525 ng/ml, FIG. 3H represents a case for unstable angina (UA), in which *P value=0.0495, Cut-off value: >0.407 ng/ml and FIG. 3I represents a case for angina pectoris (AP), in which P value=0.3088, and Cut-off value: >0.399 ng/ml. (*statistically significant)

As shown in FIGS. 3A to 3I, the patients with acute myocardial infarction showed significantly (p<0.05) higher concentrations in all three peptides as compared to those of normal people (control). Cut-off values were determined as 29.05 fmol/ml (NT-proBNP), 53.58 pg/ml (SubP), and 0.525 ng/ml (NpY) (confidence interval 95%).

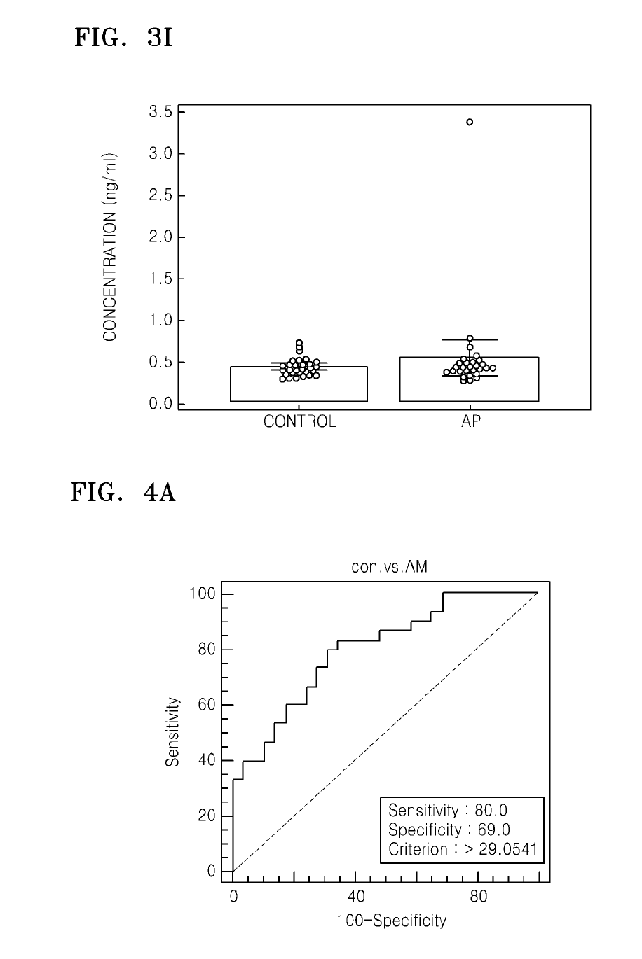

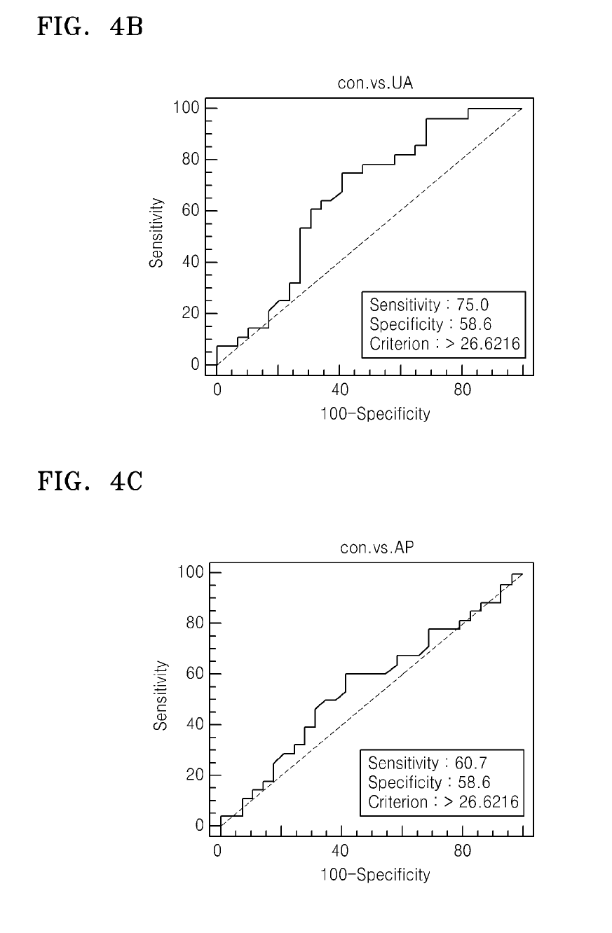

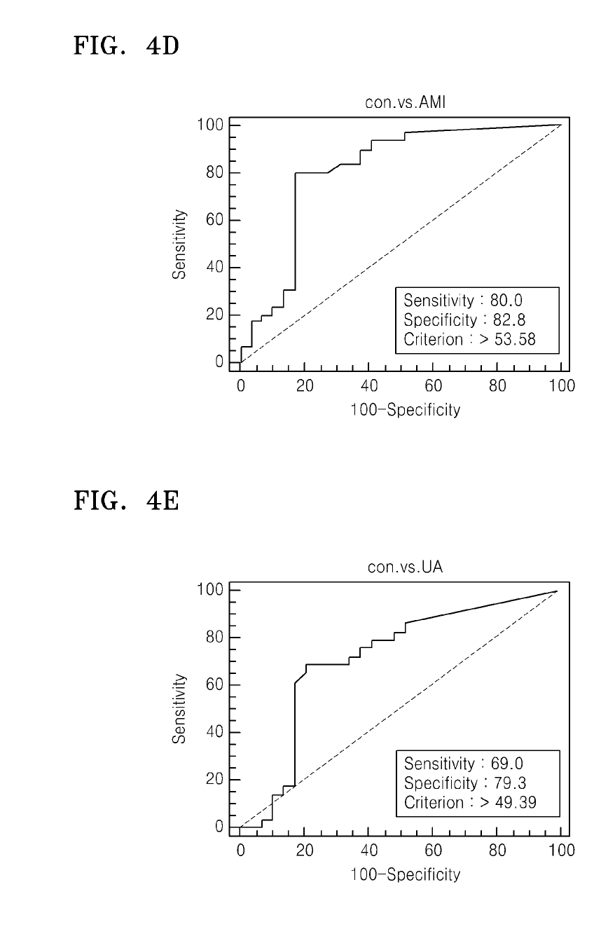

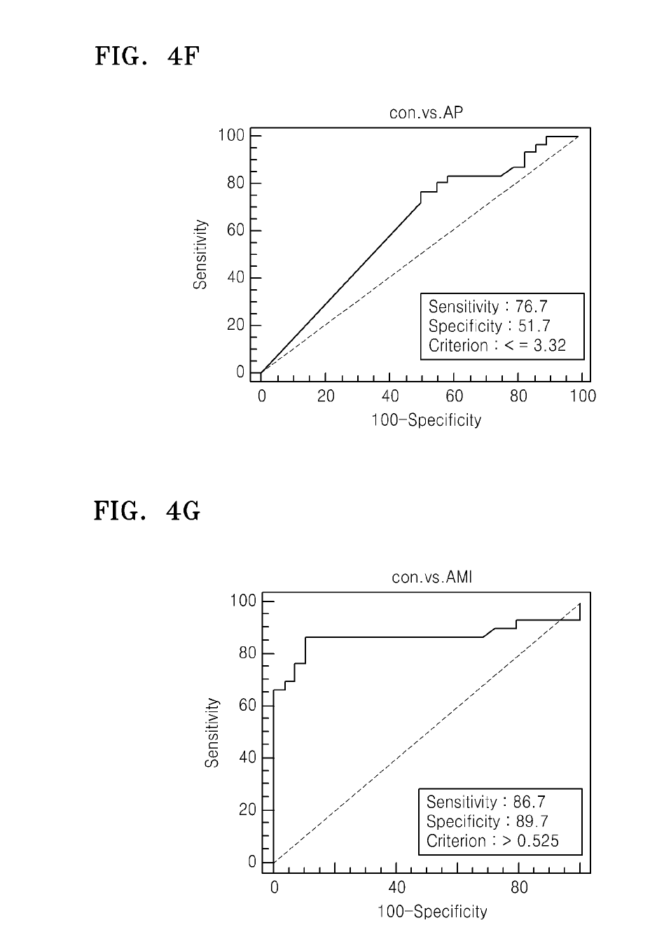

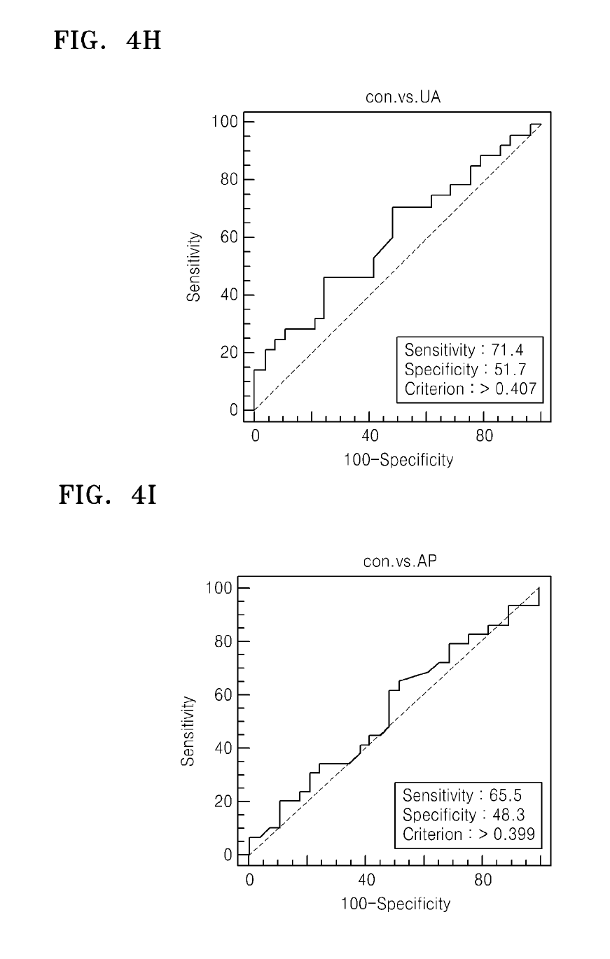

FIGS. 4A-4C, 4D-4F, and 4G-4I show graphs of a Receiver Operating Characteristic (ROC) of NT-proBNP, SubP and NpY obtained using blood samples of normal people and those of patients with a cardiovascular disease.

Referring to FIGS. 4A-4C, which relates to ‘NT-proBNP’, FIG. 4A represents a curve for acute myocardial infarction (AMI), in which Area Under the Curve(AUC)=0.80, FIG. 4B represents a curve for unstable angina (UA), in which AUC=0.66 and FIG. 4C represents a curve for angina pectoris (AP), in which AUC=0.56.

Referring to FIGS. 4D-4F, which relates to ‘SubP’, FIG. 4D represents a curve for acute myocardial infarction (AMI), in which AUC=0.80, FIG. 4E represents a curve for unstable angina (UA), in which AUC=0.71, and FIG. 4F represents a curve for angina pectoris (AP), in which AUC=0.63.

Referring to FIGS. 4G-4I, which relates to NpY, FIG. 4G represents a curve for acute myocardial infarction (AMI), in which AUC=0.87, FIG. 4H represents a curve for unstable angina (UA), in which AUC=0.61, and FIG. 4I represents a curve for angina pectoris (AP), in which AUC=0.54.

As shown in FIGS. 4A to 4I, the ROC curves obtained from the concentrations of pro-BNP, SubP, and NpY measured from the blood samples of normal people and those with a cardiovascular disease revealed that, pro-BNP had 69% specificity, 80% sensitivity, and a criterion of the cardiovascular disease was 29.05 fmol/ml; SubP had 83% specificity, 80% sensitivity, and a criterion of the cardiovascular disease was 53.58 pg/ml; and NpY had 90% specificity, 87% sensitivity, and a criterion of the cardiovascular disease was 0.525 ng/ml. AUC for each of the ROC curves was 0.80 (NT-proBNP), 0.80 (SubP) and 0.87 (NpY) (Table 3). From these results, it was confirmed that the two markers of SubP and NpY are more disease-specific and more useful diagnostic markers for acute myocardial infarction as compared with the well-known NT-proBNP.

TABLE 2

| Parameter | SubP | NpY |
|---|---|---|
| Cut-off value | 53.58 pg/ml | 0.525 ng/ml |
| AUC | 0.95 ± 0.062^{a, b} | 0.87 ± 0.056^{a, b} |
| Specificity | 83% | 90% |
| Sensitivity (95% CI) | 80% (61-92) | 87% (69-96) |

AUC: area under the curve;
CI, confidence interval;
^{a}AUC ± standard error
^{b}All p < 0.05 when comparing AUC of SubP and NpY

Sensitivity and specificity in each assay were calculated using a cut-off value of SubP and NpY, respectively.

While the present invention has been particularly shown and described with reference to exemplary embodiments thereof, it will be understood by those of ordinary skill in the art that various changes in form and details may be made therein without departing from the spirit and scope of the present invention as defined by the following claims.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition for diagnosing acute myocardial infarction comprising a preparation for measuring an amount of substance P (SubP) or neuropeptide Y (NpY).

2. The composition of claim 1, wherein the substance P comprises an amino sequence of SEQ ID NO: 1.

3. The composition of claim 1, wherein the neuropeptide Y comprises an amino sequence of SEQ ID NO: 2.

4. The composition of claim 1, wherein the preparation for measuring the amount of substance P or neuropeptide Y is an antibody specifically binding to the substance P or the neuropeptide Y, or a fragment thereof.

5. The composition of claim 4, wherein the antibody is a monoclonal or polyclonal antibody.

6. The composition of claim 1, wherein the preparation for measuring the amount of substance P or neuropeptide Y is a partial peptide having a binding domain specific to the substance P or the neuropeptide Y, or a fragment thereof.

7. A kit for diagnosing acute myocardial infarction comprising the composition of claim 1.

8. A method of diagnosing acute myocardial infarction, the method comprising:
measuring an amount of substance P or neuropeptide Y in a sample obtained from a subject;
comparing the measured amount of the substance P or the neuropeptide Y with that of a normal control group; and
determining the subject as having acute myocardial infarction if the amount of the protein in the sample obtained from the subject is higher than that of the normal control group.

9. The method of claim 8, wherein the sample obtained from the subject comprises blood, liquid bone marrow, lymph, saliva, lachrymal fluid, mucus, amniotic fluid, or a combination thereof.

10. The method of claim 8, wherein the amount of substance P or neuropeptide Y is measured using an antibody which specifically binds to the substance P or the neuropeptide Y, or a fragment thereof.

11. The method of claim 8, wherein the amount of substance P or neuropeptide Y is measured using a partial peptide having a binding domain specific to the substance P or the neuropeptide Y, or a fragment thereof.

12. The method of claim 8, wherein the amount of substance P or neuropeptide Y is measured by western blot, enzyme-linked immunosorbent assay (ELISA), radioimmunoassay (RIA), radioimmunodiffusion, Ouchterlony immunodiffusion, rocket immunoelectrophoresis, histoimmunostaining, immunoprecipitation assay, complement fixation assay, complement fixation test, fluorescence-activated cell sorting (FACS), protein chips or a combination thereof.

13. The method of claim 8, wherein the subject is diagnosed as having acute myocardial infarction when the amount of the substance P in blood is more than 53.58 pg/ml or the amount of the neuropeptide Y in blood is more than 525 pg/ml.

14. The method of claim 8, wherein the subject is diagnosed as having unstable angina when the amount of the substance P in blood is less than 53.58 pg/ml blood, and the amount of the neuropeptide Y in blood is more than 407 pg/ml but less than 525 pg/ml.

15. The method of claim 8, wherein the subject is diagnosed as not having acute myocardial infarction or unstable angina when the amount of the substance P in blood is less than 53.58 pg/ml, and the amount of the neuropeptide Y in blood is less than 407 pg/ml.

16. A kit for diagnosing acute myocardial infarction comprising the composition of claim 2.

17. A kit for diagnosing acute myocardial infarction comprising the composition of claim 3.

18. A kit for diagnosing acute myocardial infarction comprising the composition of claim 4.

19. A kit for diagnosing acute myocardial infarction comprising the composition of claim 5.

20. A kit for diagnosing acute myocardial infarction comprising the composition of claim 6.
